(19) United States
(12) Patent Application Publication
(10) Pub. No.: US 2011/0082506 A1
(43) Pub. Date: Apr. 7, 2011

(54) CERVICAL PLATE ASSEMBLY

(75) Inventors: VITO LORE, SOMERVILLE, MA (US); CHRISTOPHER A. CHANG, BEVERLY, MA (US); CRAIG HENSHAW, CHARLESTOWN, MA (US); KINGSLEY CHIN, Hallandale, FL (US); MICHAEL CORDONNIER, MEMPHIS, TN (US); BURL DANIEL, NEW ALBANY, MS (US)

(73) Assignee: SPINEFRONTIER, INC, BEVERLY, MA (US)

(21) Appl. No.: 12/894,776

(22) Filed: Sep. 30, 2010

Related U.S. Application Data
(60) Provisional application No. 61/248,148, filed on Oct. 2, 2009.

(51) Int. Cl. A61B 17/88; A61B 17/70; A61B 17/56

(52) U.S. Cl. 606/279; 606/246; 606/104

(57) ABSTRACT

An implantable cervical plate assembly for stabilization of two adjacent spinal vertebras includes a cervical plate and one or more bone fasteners. The cervical plate comprises an elongated asymmetric body having a first straight side surface, a second contoured side surface opposite to the first side surface, front and back surfaces and top and bottom surfaces. The elongated asymmetric body comprises one or more through-openings extending from the front surface to the back surface of the elongated asymmetric body. The one or more bone fasteners are configured to be inserted through the one or more through-openings, respectively, and to be attached to locations in the spinal vertebras, thereby attaching the cervical plate to the spinal vertebras. The through-openings comprise a first diameter at the front surface of the elongated body, a second diameter at the back surface of the elongated body and a third diameter in the area between the front and back surfaces of the elongated body. The first diameter is smaller than the third diameter, thereby forming a lip at the top of the through-openings. The third diameter is larger than the second diameter and the first diameter is larger than the second diameter, thereby forming a groove within the perimeter of the inner wall of the through-openings. The bone fasteners comprise a threaded main body and a head. The threaded main body comprises threads for engaging the spinal vertebras and the head comprises one or more flexible structures configured to be flexed and inserted into the groove and then unflex and remain captured within the groove.

### CROSS REFERENCE TO RELATED CO-PENDING APPLICATIONS

This application claims the benefit of U.S. provisional application Ser. No. 61/248,148 filed Oct. 2, 2009 and entitled “CERVICAL PLATE ASSEMBLY”, the contents of which are expressly incorporated herein by reference.

### FIELD OF THE INVENTION

The present invention relates to a cervical plate assembly, and in particular to a cervical plate assembly including an asymmetric plate and screws for attaching the plate to the bone.

### BACKGROUND OF THE INVENTION

Spine fixation assemblies are used to stabilized diseased or surgically removed vertebral elements. Several prior art spine fixation assemblies utilize rods and/or plates as connecting and stabilization elements between the vertebral elements. The rods and/or plates are usually secured to vertebral bones via screws. In situations and/or spinal locations where the vertebral elements are allowed to move after the rod or plate is attached, stresses associated with this motion or stresses due the motion of adjacent vertebral elements often cause the screws to disengage from the rod or plate and finally from the vertebral elements. Accordingly, there is a need for a locking mechanism that would prevent such a disengagement of the screws from the rod or plate and the vertebral elements.

### SUMMARY OF THE INVENTION

The present invention relates to a system and method for a cervical plate assembly and in particular to a cervical plate assembly that includes an asymmetric bone plate and screws attaching the plate to vertebral elements. The screws include a self-contained locking mechanism that prevents accidental disengagement of the screws due to stresses after they have been attached to the vertebral elements.

In general, in one aspect, the invention features an implantable cervical plate assembly for stabilization of two adjacent spinal vertebras including a cervical plate and two or more bone fasteners. The cervical plate comprises an elongated asymmetric body having a first straight side surface, a second contoured side surface opposite to the first side surface, front and back surfaces and top and bottom surfaces. The elongated asymmetric body comprises two or more through-openings extending from the front surface to the back surface of the elongated asymmetric body. The two or more bone fasteners are configured to be inserted through the two or more through-openings, respectively, and to be attached to two or more locations in the two adjacent spinal vertebras, respectively, thereby attaching the cervical plate to the spinal vertebras. The through-openings comprise a first diameter at the front surface of the elongated body, a second diameter at the back surface of the elongated body and a third diameter in the area between the front and back surfaces of the elongated body. The first diameter is smaller than the third diameter, thereby forming a lip at the top of the through-openings. The third diameter is larger than the second diameter and the first diameter is larger than the second diameter, thereby forming a groove within the perimeter of the inner wall of the through-openings. The bone fasteners comprise a threaded main body and a head. The threaded main body comprises threads for engaging the spinal vertebras and the head comprises one or more flexible structures configured to be flexed and inserted into the groove and then unflex and remain captured within the groove.

Implementations of this aspect of the invention may include one or more of the following features. The through-openings comprise an oval-shaped perimeter at the back surface and the oval-shaped perimeter comprises two parallel straight sides and two opposite curved sides. The distance between the two parallel straight sides is smaller than the major diameter of the threads of the bone fasteners and the distance between the curved sides is equal to or larger than the major diameter of the threads of the bone fasteners. The bone fastener head comprises a cylindrical main body and the one or more flexible structures comprise one or more flexible arms extending tangentially from the outer side surface of the cylindrical main body and curving counter-clockwise around the cylindrical main body. The diameter of the bone fastener head including the flexible arms in the unflexed position is larger than the first diameter of the through openings and the flexible arms are configured to flex inward toward the outer side surface of the cylindrical main body when they come in contact with the lip while the bone fastener is rotated clock-wise to be driven into the vertebras and then the flexible arms unflex once they are below the lip. The bone fastener head comprises an opening extending into the threaded main body and the opening comprises an inner surface having six inward protruding lobes and a bottom having six grooves. The assembly may further include a driver tool. The driver tool comprises an elongated shaft, a handle attached to the proximal end of the elongated shaft and a bone fastener-engaging component attached to the distal end of the elongated shaft. The bone fastener-engaging component comprises one or more structures that complement and engage at least one of the grooves and lobes of the bone fastener opening. The structures of the fastener-engaging component comprise four lobes that complement and engage four of the six lobes of the bone fastener opening and two opposite tubular protrusions configured to be positioned and engage two opposite located grooves of the bone fastener opening. The fastener-engaging component comprises a driver and a locking sleeve. The driver comprises an elongated cylindrical body having the structures at its distal end and a slot extending along the driver tool axis. The cylindrical body flexes and snaps into the bone fastener opening and the locking sleeve is configured to move down and lock the driver into the bone fastener opening. The locking sleeve comprises a tubular cylindrical body and a central blade. The tubular cylindrical body is dimensioned to fit and slide over the driver cylindrical elongated body and the central blade is configured to be placed within the driver slot. The structures of the bone fastener-engaging component may be outer threads configured to engage inner threads in the opening of the bone fastener. The flexible arms comprise curved, angled or beveled outer surfaces and the flexible arms outer surfaces cooperate with matching outer surfaces of the lip. The bone fastener head comprises an opening extending into the threaded main body and the opening comprises pentagonal, hexagonal or octagonal geometric shape, or inner threads. The cervical plate may further comprise one or more elongated openings configured to support bone graft material. The bone fasteners may further comprise a tapered portion extending between the threaded main body and the head and in this case the parallel straight sides of the through-openings cut into the diameter of the tapered portion for a tighter secure lock and fit. The through-openings may further include laser-etched ridges extending perpendicular to said groove. The back surface of the cervical plate may have a roughened texture.

In general in another aspect the invention features an implantable cervical plate assembly for stabilization of two adjacent spinal vertebras including a cervical plate and two or more bone fasteners. The cervical plate comprises an elongated body having first and second side surfaces, front and back surfaces and top and bottom surfaces and the elongated body comprises two or more through-openings extending from the front surface to the back surface of the elongated body. The two or more bone fasteners are configured to be inserted through the two or more through-openings, respectively, and attached to two or more locations in the two adjacent spinal vertebras, respectively, thereby attaching the cervical plate to the spinal vertebras. The bone fasteners comprise a threaded main body and a head and the threaded main body comprises threads for engaging the spinal vertebras. The through-openings comprise a perimeter dimensioned and shaped to match and complement the shape of the bone fastener head. The through-openings further comprise two opposite radially extending slots and two grooves positioned adjacent to the slots within the inner wall of the through openings, respectively. The head comprises two opposite radially protruding tubular extensions dimensioned and configured to be inserted into the two opposite radially extending slots and then rotated and captured within the two adjacent grooves, respectively.

In general in another aspect the invention features a bone fastener driver tool including an elongated shaft, a handle attached to the proximal end of the elongated shaft and a bone fastener-engaging component attached to the distal end of the elongated shaft. The bone fastener-engaging component comprises one or more structures that complement and engage at least one of grooves and protruding lobes within an opening of a bone fastener. The bone fastener-engaging component further comprises a driver and a locking sleeve. The driver comprises an elongated cylindrical body having the structures at its distal end and a slot extending along the driver tool axis. The cylindrical body flexes and snaps into the bone fastener opening and the locking sleeve is configured to move down and lock the driver into the bone fastener opening. The structures of the fastener-engaging component comprise four lobes that complement and engage four lobes in the bone fastener opening and two opposite tubular protrusions configured to be positioned and engage two opposite located grooves in the bone fastener opening. The locking sleeve comprises a tubular cylindrical body and a central blade and the tubular cylindrical body is dimensioned to fit and slide over the driver cylindrical elongated body and the central blade is configured to be placed within the driver slot. The structures of the fastener-engaging component may be outer threads configured to engage inner threads in the bone fastener opening.

In general in another aspect the invention features a method for stabilizing two adjacent spinal vertebras, including providing a cervical plate and then inserting two or more bone fasteners through two or more through-openings of the cervical plate, respectively, and attaching them to two or more locations in the two adjacent spinal vertebras, respectively, thereby attaching the cervical plate to the spinal vertebras. The cervical plate comprises an elongated asymmetric body having a first straight side surface, a second contoured side surface opposite to the first side surface, front and back surfaces and top and bottom surfaces. The elongated asymmetric body comprises two or more through-openings extending from the front surface to the back surface of the elongated asymmetric body. The through-openings comprise a first diameter at the front surface of the elongated body, a second diameter at the back surface of the elongated body and a third diameter in the area between the front and back surfaces of the elongated body. The first diameter is smaller than the third diameter, thereby forming a lip at the top of the through-openings. The third diameter is larger than the second diameter and the first diameter is larger than the second diameter, thereby forming a groove within the perimeter of the inner wall of the through-openings. The bone fasteners comprise a threaded main body and a head. The threaded main body comprises threads for engaging the spinal vertebras and the head comprises one or more flexible structures configured to be flexed and inserted into the groove and then unflex and remain captured within the groove.

### BRIEF DESCRIPTION OF THE DRAWINGS

Referring to the figures, wherein like numerals represent like parts throughout the several views:

FIG. 1 is a perspective view of a cervical plate assembly;

FIG. 2A is a perspective view of the cervical plate of FIG. 1;

FIG. 2B is a side view of the cervical plate of FIG. 2A;

FIG. 3 is a top view of the cervical plate of FIG. 2A;

FIG. 4A is a side view of end 111b of the cervical plate of FIG. 2B;

FIG. 4B is a cross-sectional view of the cervical plate along line 113;

FIG. 5 is a perspective view of the screw of FIG. 1;

FIG. 6A is a top view of the screw of FIG. 5;

FIG. 6B is a side view of the screw of FIG. 5;

FIG. 7 is a cross-sectional view of the cervical plate assembly;

FIG. 8A is a detailed side view of area A in FIG. 7

FIG. 8B is a detailed top view of area A in FIG. 7;

FIG. 9 is top perspective view of the cervical plate assembly of FIG. 7;

FIG. 10 is a cross-sectional view depicting an angular placement of a the screw within an opening of the cervical place;

FIG. 11 depicts a two-component driver tool;

FIG. 11A depicts a driver tool end in the unlocked position;

FIG. 11B depicts the driver tool end of FIG. 11A in the locked position;

FIG. 11C is a cross-sectional view of FIG. 11A;

FIG. 11D is a cross-sectional view of FIG. 11B;

FIG. 11E is a detailed view of the lower end of the driver tool in the locked position;

FIG. 11F is an exploded view of FIG. 11C;

FIG. 11G is a detailed bottom view of the driver 210;

FIG. 11H is a detailed bottom view of the driver 210 with the lowered blade 226;

FIG. 12 is an exploded view of FIG. 11A;

FIG. 13 depicts detailed views of the driver lower end and the locking sleeve end;

FIG. 14A depicts a driver tool end for removing a bone screw;

FIG. 14B is an exploded view of the driver tool end of FIG. 14A;

FIG. 15A is another embodiment of a bone screw with a self-contained locking mechanism;

FIG. 15B is a partial view of the cervical plate with an opening that cooperates with the bone screw of FIG. 15A;

FIG. 16 is another embodiment of the cervical plate;

FIG. 17 is another embodiment of the cervical plate; and

FIG. 18 is another embodiment of the cervical plate.

### DETAILED DESCRIPTION OF THE INVENTION

The present invention relates to a system and method for a cervical plate assembly that includes an asymmetric bone plate and screws attaching the plate to vertebral elements. The screws include a self-contained locking mechanism that prevents accidental disengagement of the screws due to stresses after they have been attached to the vertebral elements.

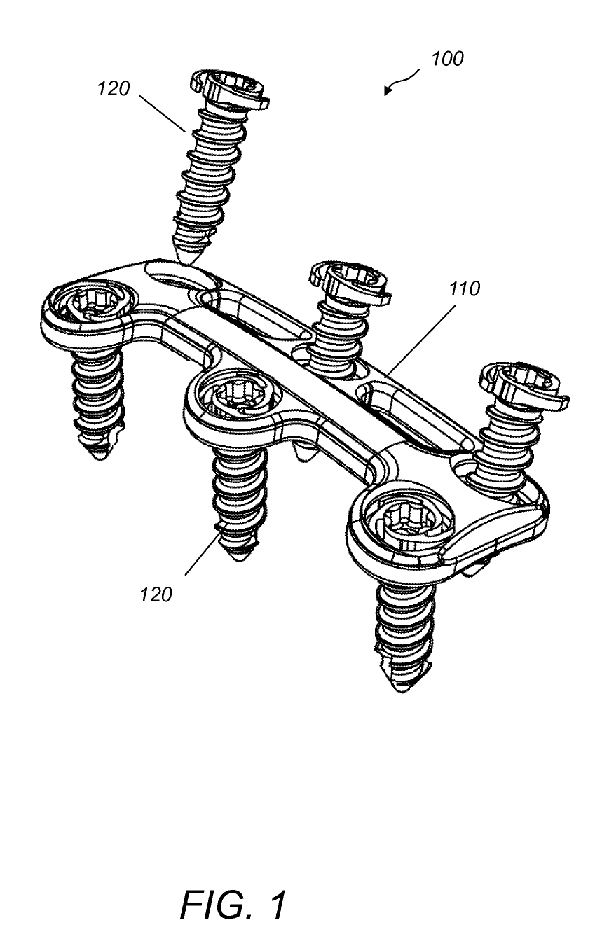

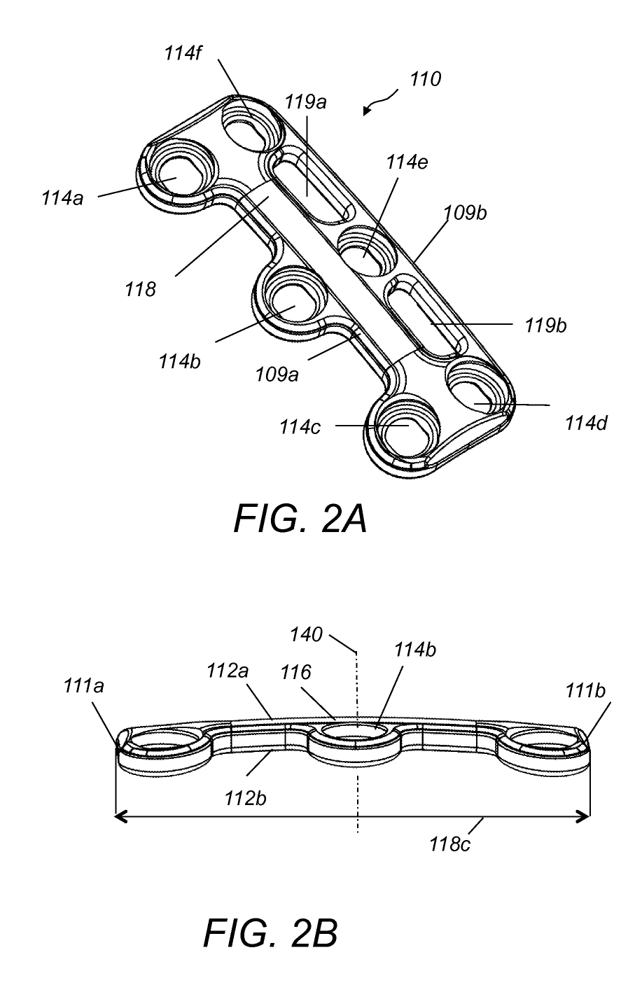

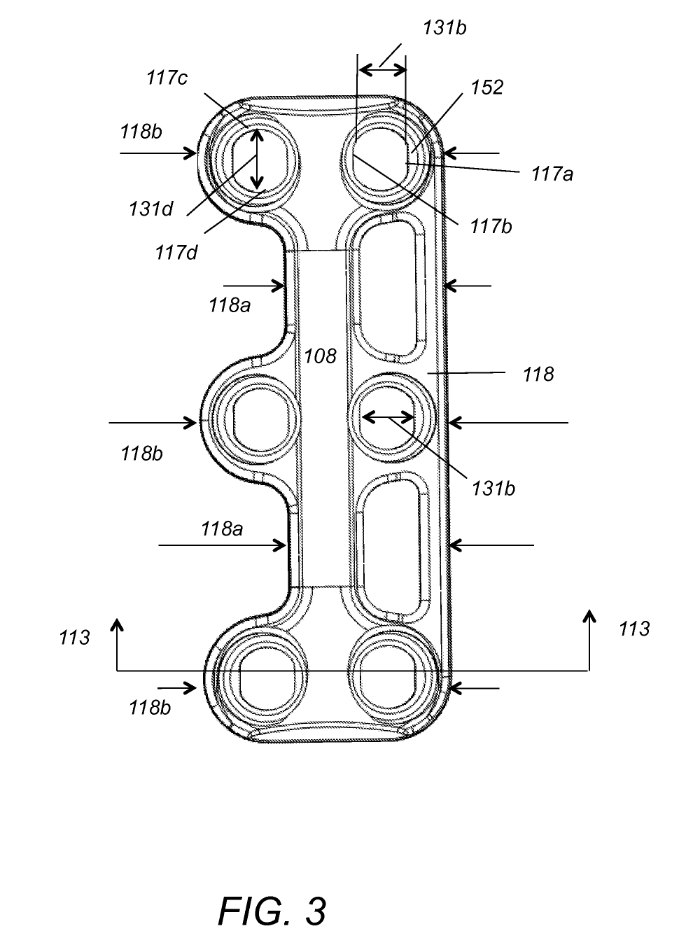

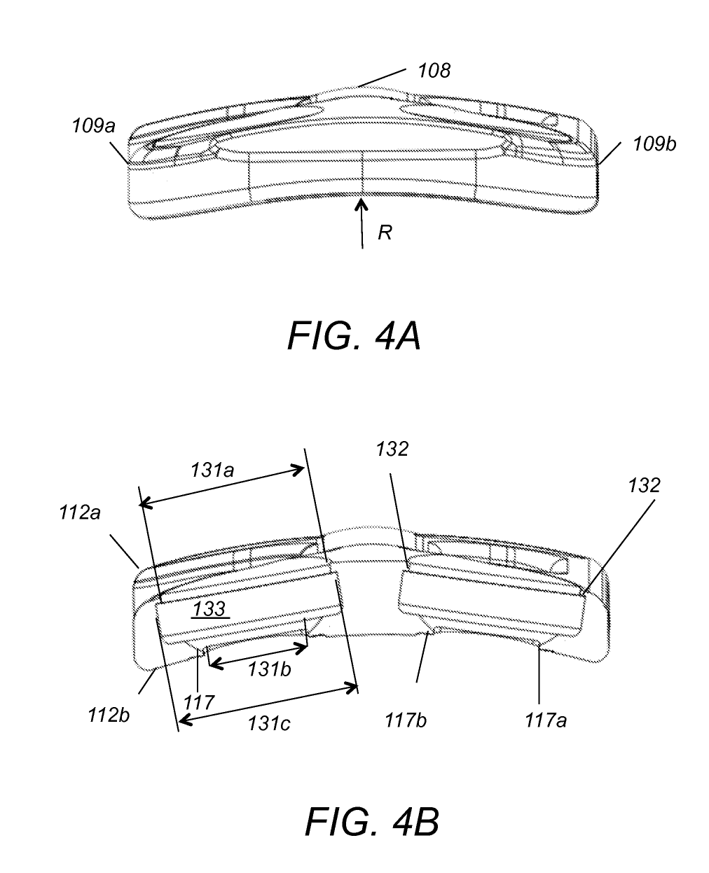

Referring to FIG. 1, cervical plate assembly 100 includes a cervical plate 110 and screws 120. Cervical plate 110 is a two-level bone plate configured to stabilize three adjacent vertebras (not shown). Referring to FIG. 2A, FIG. 2B, FIG. 3, FIG. 4A and FIG. 4B, plate 110 includes an elongated asymmetric body 118 that has six through-openings 114a-114f extending from the top surface 112a to the bottom surface 112b of body 118. Body 118 has one side 109b that is straight and an opposite side 109a that is contoured around the openings 114a-114c. The width 118a of plate 110 in the area inbetween openings 114a, 114b and inbetween 114b, 114c is smaller than the width 118b in the areas across openings 114a, 114f at the end 111a of the plate, across openings 114c, 114d at the end 111b of the plate and across openings 114b, 114e at the center 116 of the plate. In one example, body 118 has a length 118c of 43 millimeters, a width 118a of 13 millimeters and a width 118b of 17 millimeters. There are also two additional through-openings 119a, 119b arranged along the straight side 109b of the plate between two adjacent main openings 114f, 114e and 114e, 114d, respectively. The reduced width 118a of the plate due to the contoured side 109a and the presence of openings 119a, 119b along the straight side 109b help improve the line of sight. Openings 119a, 119b are also used for inserting bone graft material. Cervical plate 110 is also curved along its width and is thicker along the center 108 relative to the sides 109a, 109b. In one example, the plate thickness at the center is 2.55 millimeters, the width at the sides is 2.3 millimeters and the curvature R along its width 27 millimeters. The increased thickness along the center 108 provides stability and additional strength. The overall plate thickness is kept at a minimum level in order to maintain a low profile and the overall contour of the plate is configured to provide improved anatomical interface. Cervical plate ends 111a, 111b are chamfered to minimize damage of the adjacent soft tissue. Through-openings 114a-114f receive the screws 120, which are used to attach the plate 110 to the vertebras. Openings 114a-114f have an essentially circular perimeter at the top surface 112a of the plate. The diameter 131a of each opening 114a-114f near the top surface 112a is larger than the diameter 131b near the bottom surface 112b, as shown in FIG. 4B. Both top and bottom diameters 131a, 131b are smaller than the diameter 131c at the center of the opening. In one example, diameter 131a is 6 millimeters, diameter 131b is 4.20 millimeters and diameter 131c is 6.4 millimeters. A lip 132 is formed around each opening 114a-114f near the top surface 112a. Lip 132 is designed to interface with flexible arms 121a-121c extending from the screw head 122 and thereby to lock the screw 120 onto the plate 110, as will be explained below. Openings 114a-114f have a chamfered bottom portion 117, as shown in FIG. 4B. Chamfered bottom portion 117 allows the screws 120 to assume variable trajectory and angled orientation when engaged in the vertebral bone, as shown in FIG. 10. In some embodiments, polyaxial screws 120 are used and the chamfered bottom 117 allows them to be positioned at a desired angular orientation 146 prior to being locked. The bottom portion 117 of the openings 114a-114f is oval-shaped and has two parallel straight sides 117a, 117b and two opposite curved sides 117c, 117d. The distance between the two parallel straight sides 117a, 117b (width of the opening) 131b is smaller than the major diameter 91 of the threaded portion 124 of the screw 120 and equal or larger than the minor diameter 92 of the threaded portion 124. The distance 131d between the curved sides 117c and 117d of the opening (diameter) is larger or equal to the major diameter 91 of the threaded portion 124 of the screw. The oval-shaped structure of the bottom portion 117 of openings 114a-114f cooperates with the screw threads 124a to allow the screw 120 to move downward or upwards through the opening when the screw 120 is rotated and prevents backing out or moving forward of the screw 120 when the screw is pushed up or down, respectively. Since the width 131b of the opening at the bottom portion 117 is smaller than the major diameter 91 of the threaded portion 124 of the screw 120 and the diameter 131d is larger or about the same size as the major diameter 91 of the threaded portion 124, the screw threads 124a move through the opening as they are rotated clock-wise only when they are in line with the diameter 131d. Once the screw threads 124a pass below the bottom portion 117 of the opening, they cannot be accidentally pushed straight up because they will hit the straight parallel sides 117a, 117b of the oval-shaped opening, whose spacing 131d is smaller than the major diameter 91 of the screw. This “threading” of the screw 120 through the oval-shaped opening (i.e. “captive geometry”) of the bottom portion 117 of the plate 110 locks the screw 120 to the plate 110 and prevents accidental backing out of the screw 120. Furthermore, screw 120 includes a tapered portion (angled sides 125a, 125b) and at this tapered portion the straight parallel sides 117a, 117b cut into the diameter of the tapered portion for a tighter secure lock and fit.

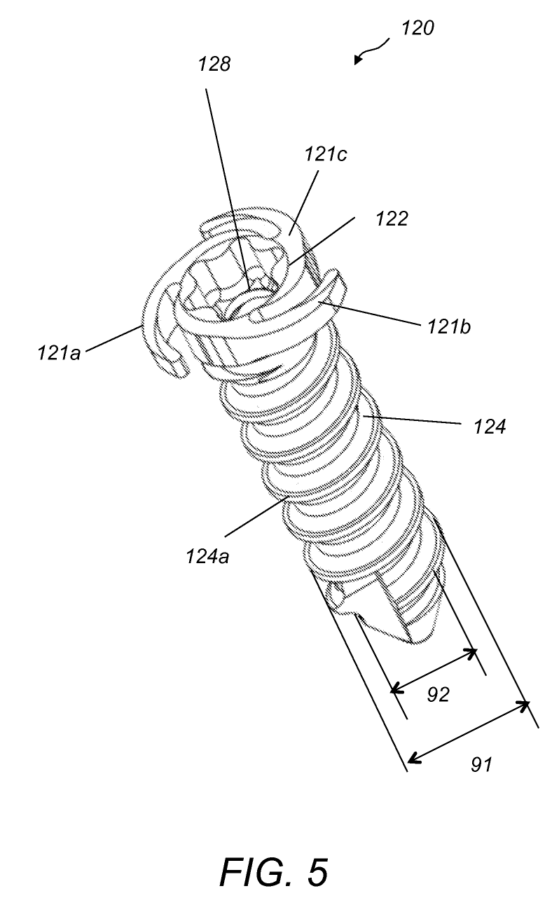

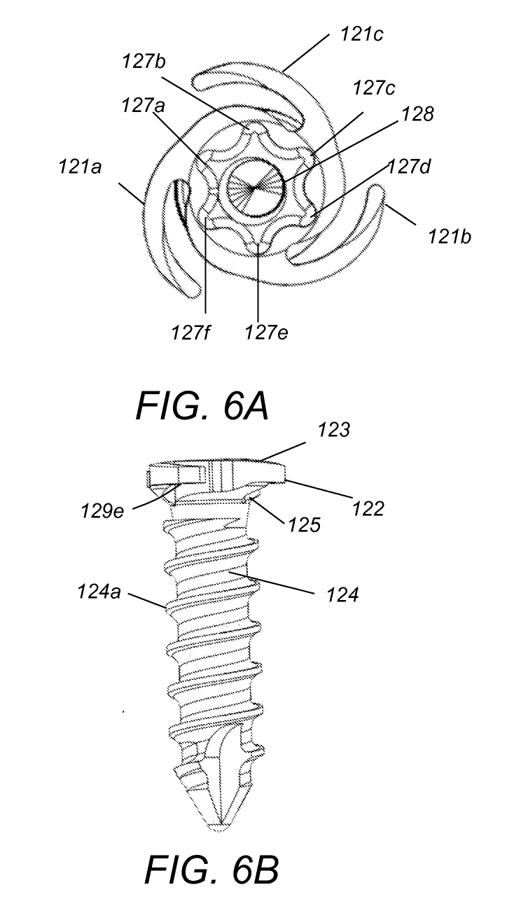

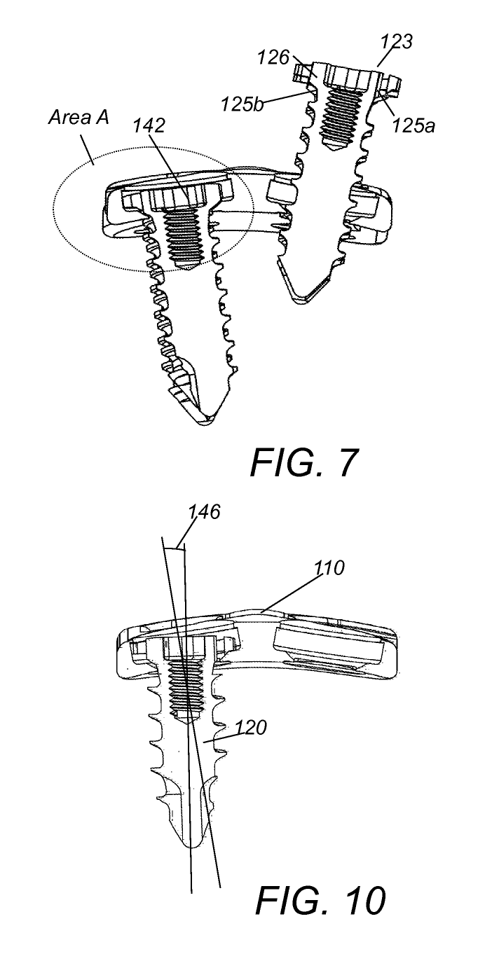

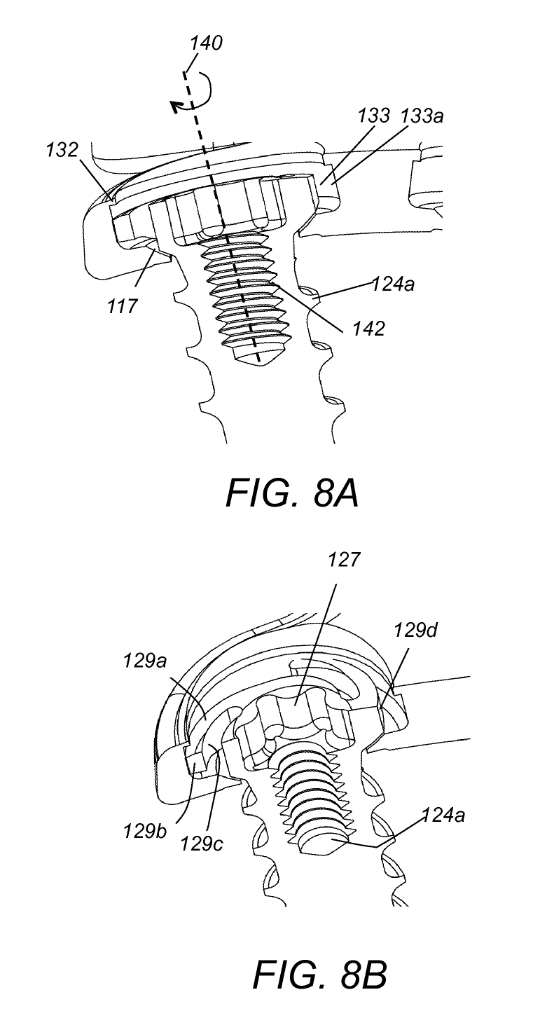

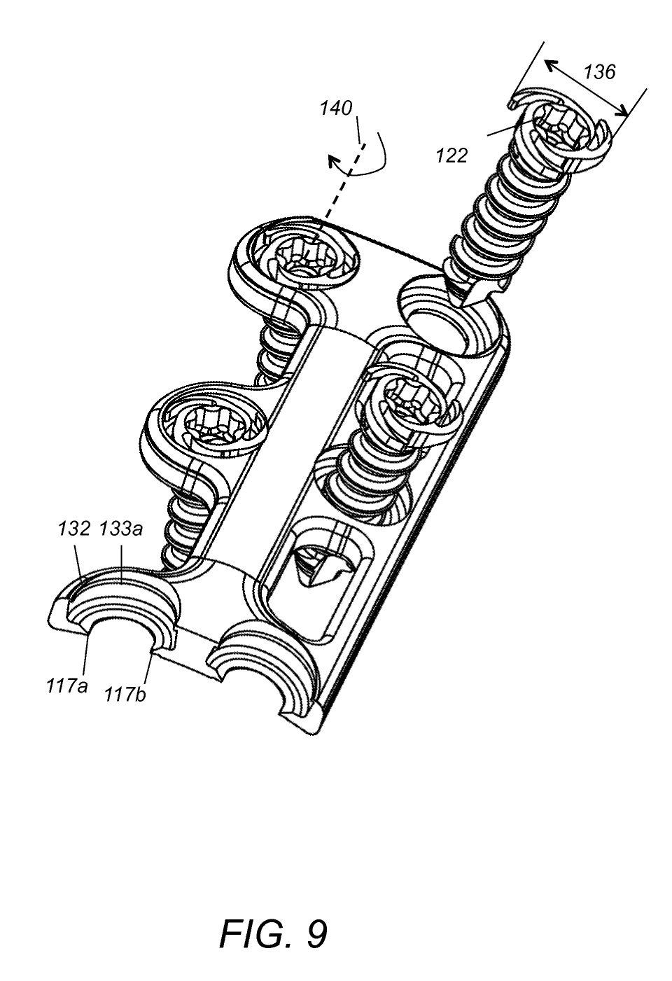

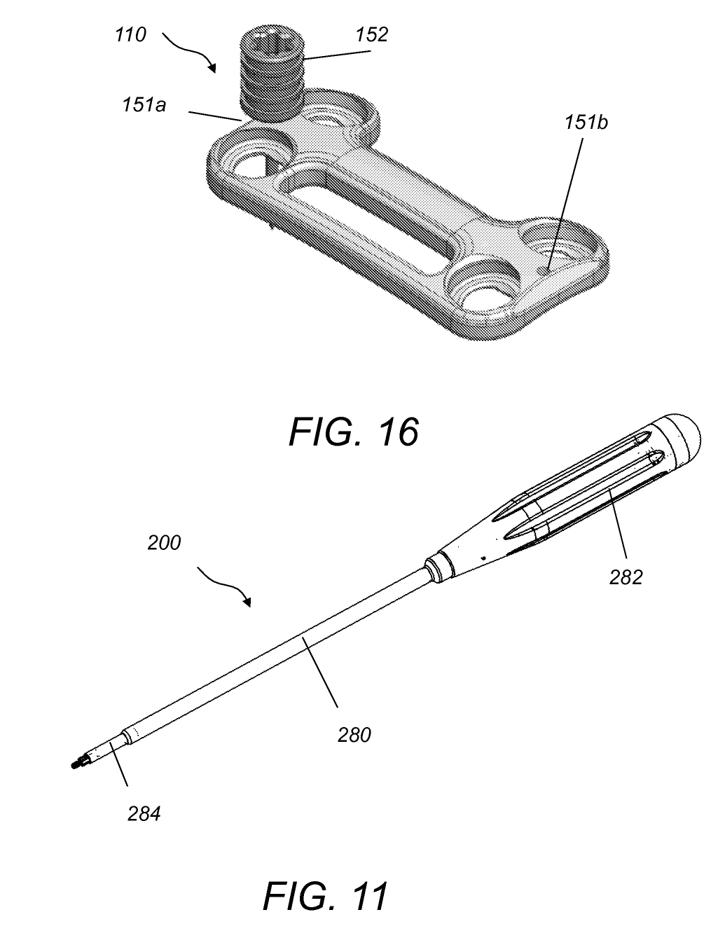

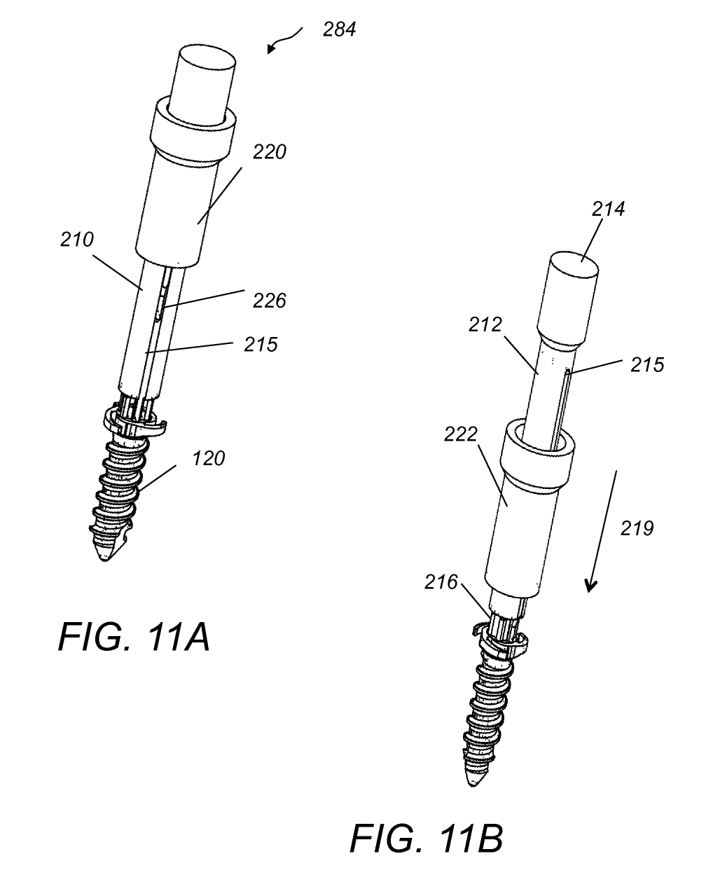

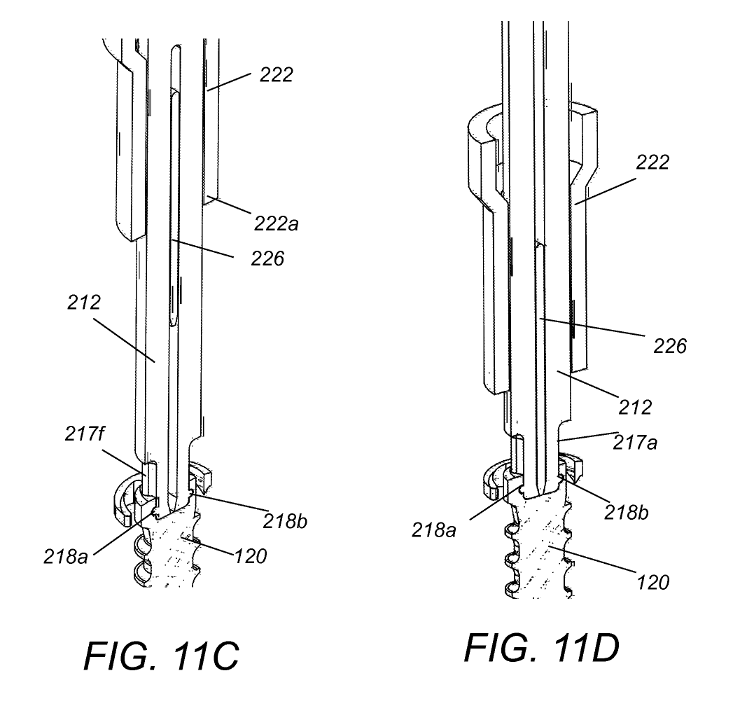

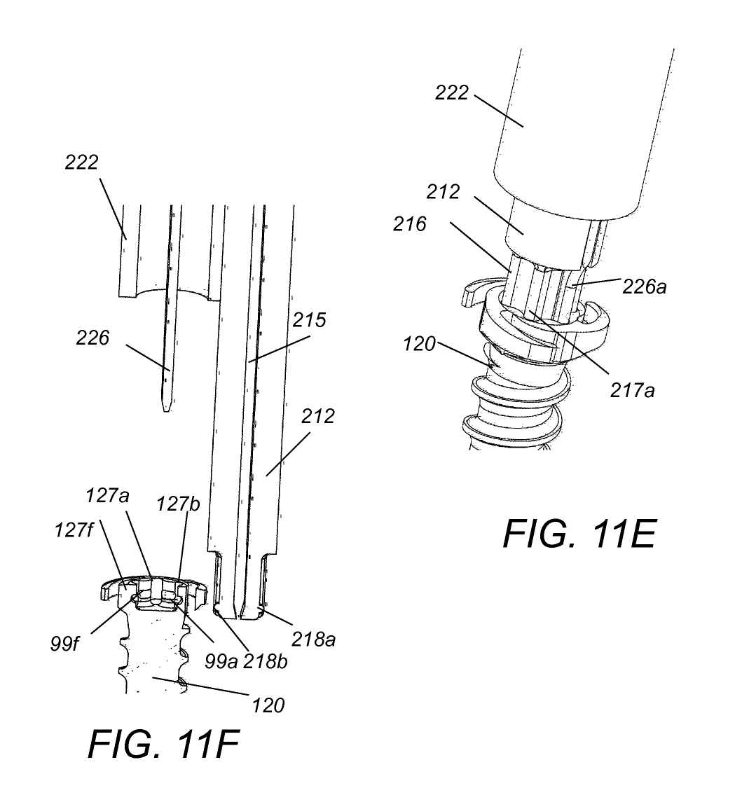

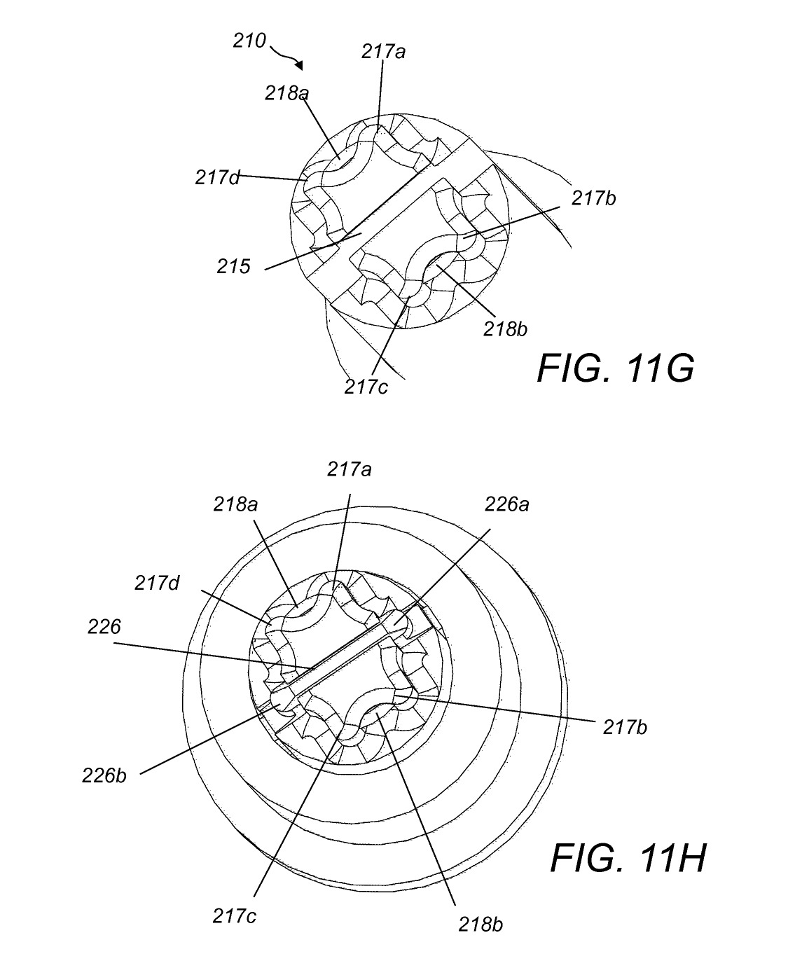

Referring to FIG. 5 to FIG. 10, bone screw 120 has a threaded main body 124 and a head 122. Main body 124 includes threads 124a for engaging the vertebral bone. Head 122 has a flat top 123, a cylindrical center 126 and a tapered portion 125 with angled bottom sides 125a, 125b, as shown in FIG. 7. Top 123 includes an opening 128 extending into the main body 124. Opening 128 has six lobes 127a-127f, and at the bottom between two adjacent lobes six grooves 99a-99f are formed, as shown in FIG. 11F. As will be explained later, the geometry of opening 128 interfaces with the geometry of a screw engaging component 284 to lock a driver tool 200 into the opening 128, as shown in FIG. 11B. Three flexible arms 121a-121c extend tangentially from the outer side of the cylindrical center 126 and curve around the center 126. The effective diameter 136 of the screw head 122 including the arms 121a-121c in the unflexed position is larger than the top diameter 131a of openings 114a-114f, shown in FIG. 9. Arms 121a-121c flex inward toward the central axis 140 when they come in contact with lip 132 of the openings 114a-114f while the screw 120 is being rotated clock-wise to be driven into the vertebral body. The effective diameter of the screw head 122 including the arms 121a-121c in the inward flexed position is smaller than the top diameter 131a of openings 114a-114f, and this allows the screw head 122 including the arms 121a-121c to move below the lip 132. Once the arms 121a-121c are below the lip 132 they expand back up to their unflexed position within the space 133 formed in the opening 114a between the lip 132 and the chamfered sides at the bottom portion 117 of the opening.

Once the entire screw head 122 is in place within space 133, the lip 132 prevents the screw head from accidentally moving up (i.e., backing out) from space 133 due to stresses applied during spinal motion. In cases where the mounted screw is rotated counter-clockwise, arms 121a-121c hit the lip 132 and sidewall 133a and flex outward away from the central axis 140, thereby increasing the effective diameter of the screw head so that it is even larger than the top diameter 131a. This outward flexing of the arms 121a-121c prevents the screw head 122 from accidentally moving up and out of space 133. The surgeon may pull out the screw with a driver tool, as will be described below.

In operation, plate 110 is attached to the vertebras with the screws 120. During the driving in of the screws into the selected vertebral locations, the screw threads 124a cooperate with the “captive geometry” at the bottom portion of the plate 117 and the flexible arms 121a-121c are flexed inward and move in space 133 where they expand back up to their unflexed state. The combination of these two mechanisms, i.e., “threading” the screw 120 though the bottom portion 117 of the plate 110 and positioning and locking of the flexible arms 121a-121c in space 133, lock the screw 120 onto the plate 110 and prevent accidental disengagement due to stresses generated during motion.

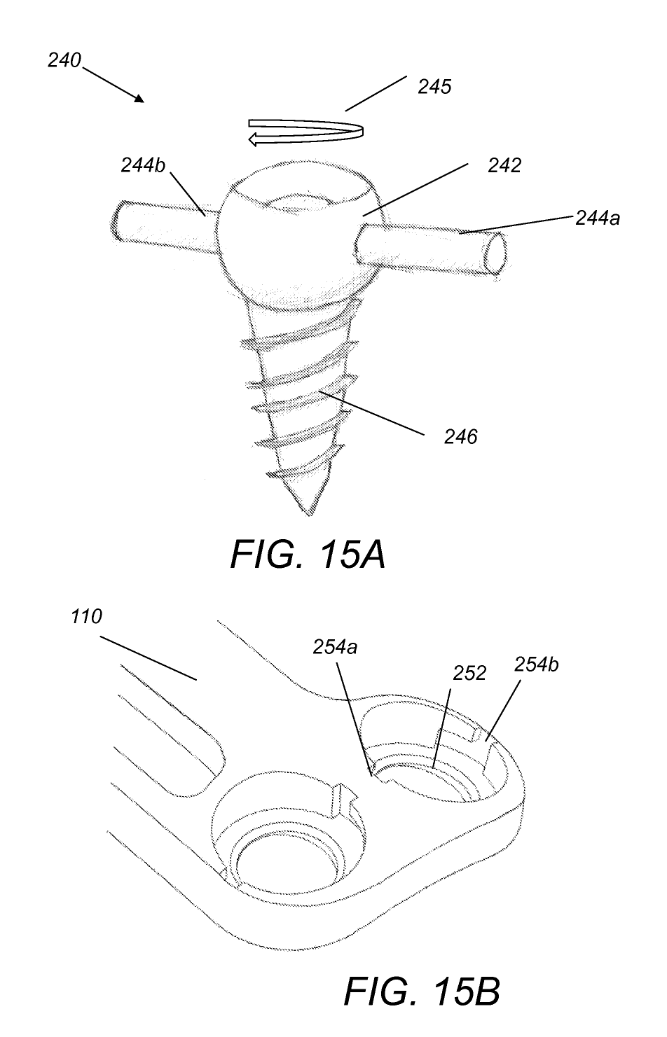

Referring to FIG. 15A, in another embodiment bone screw 240 includes a threaded main body 246 and a spherical head 242 having two horizontally extending protrusions 244a, 244b. Protrusions 244a, 244b extend outward radially from the spherical head 242. Referring to FIG. 15B, opening 252 in the cervical plate 110 includes two diametrically opposite slots 254a, 254b dimensioned and shaped to receive the protrusions 244a, 244b, respectively. Placing protrusions 244a, 244b in the slots 254a, 254b, respectively, and rotating the spherical head in the direction 245 locks the screw 240 in the cervical plate opening 252 and prevents accidental removal of the screw.

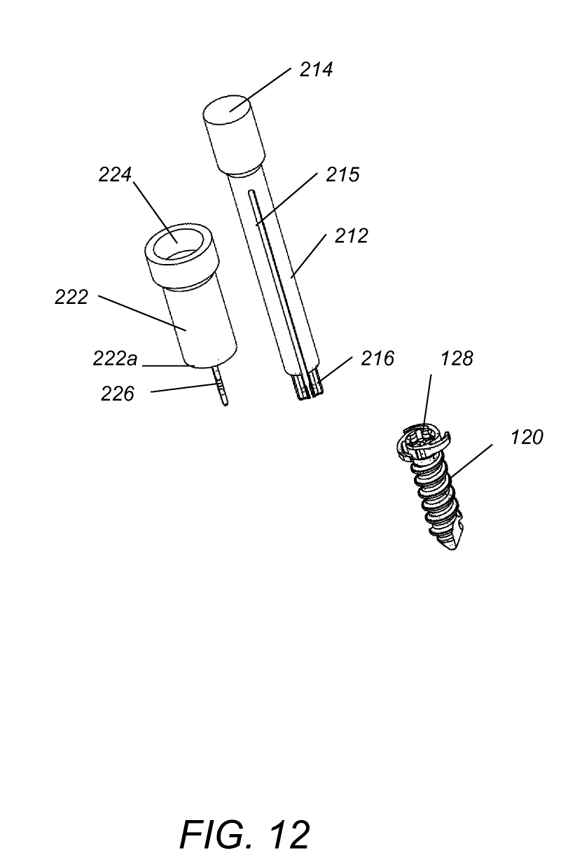

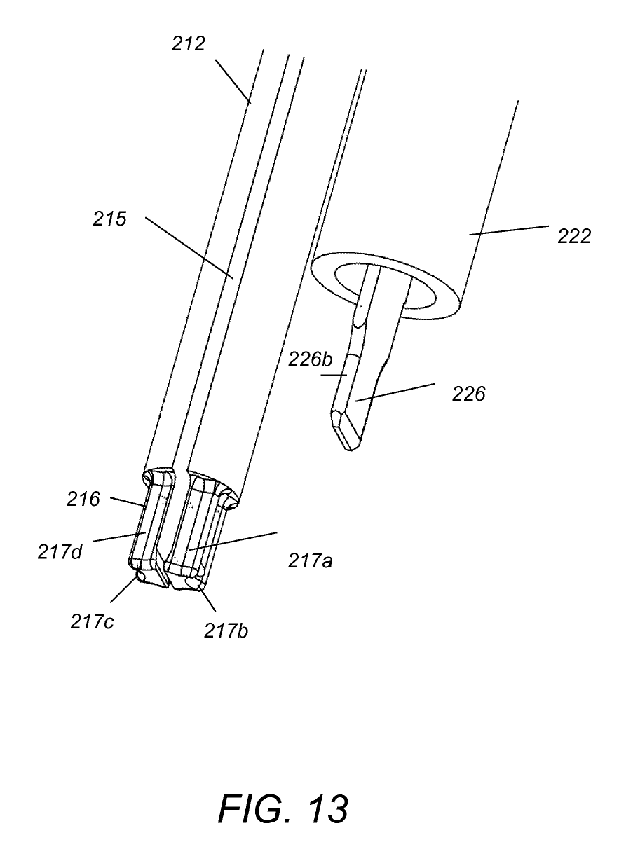

Referring to FIG. 11 to FIG. 13, a two-component tool 200 is used to drive screw 120 through the openings 114a-114f of the cervical plate 100 into the bone. Tool 200 includes an elongate shaft 280 having a handle 282 at its proximal end and a screw engaging component 284 at its distal end. Screw engaging component 284 includes a driver 210 and a locking sleeve 220. Driver 210 has an elongated cylindrical body 212 with a cylindrical top 214 and a driver end 216. The driver end 216 includes four lobes 217a-217d that match and interface with four of the six lobes 127a, 127c, 127d, 127f of opening 128 in the screw top 123, respectively. Driver end 216 also includes two tubular protrusions 218a, 218b positioned between lobes 217a, 217d and 217d, 217c, respectively. Protrusions 218a, 218b fit within opposite located grooves 99a and 99d formed between adjacent lobes in opening 128. The interfacing of the driver end geometry with the screw head opening 128 geometry engages the driver 210 to the screw head 122. In this engaged position, the driver is used to rotate screw 120 clockwise or counter-clockwise. An elongated slot 215 extends along the length of the cylindrical body 212 through its center and allows the body 212 to flex and snap into opening 128 of the screw head. Once the driver end 216 is snapped into opening 128, the locking sleeve 220 is moved down to lock the driver 210 into the opening 128 of the screw head. Locking sleeve 220 has a cylindrical body 222 with a diameter larger than the diameter of the cylindrical body 212 of the driver. Cylindrical body 222 has a central opening 224 extending the entire length of body 222 and a central blade 226 extending from about the middle of body 222 toward and past the lower end 222a of body 222. Driver 210 is inserted into the central opening 224 of the locking sleeve 220 and slot 215 is aligned with and placed over blade 226, as shown in FIG. 11A and FIG. 11C. After placing the driver end 216 into the screw opening 128, the locking sleeve 220 is moved down in the direction 219 so that the blade 226 is positioned in the slot area of the driver end 216, shown in FIG. 11B and FIG. 11D. The two parallel sides 226a, 226b of blade 226 protrude through the sides of slot 215, as shown in FIG. 11E. The protruding blade sides 226a, 226b interface with two opposite lobes 127b, 127e in opening 128, respectively. The placing of the blade 226 within the slot 215 in the screw head opening 128 prevents the lower end of body 212 from flexing and thereby locks the driver 210 within the screw head opening 128. The locked driver 210 is then used to rotate clockwise or counter-clockwise screw 120 into or out of the desired bone location, respectively, and to drive or pull the screw 120 in or out of place.

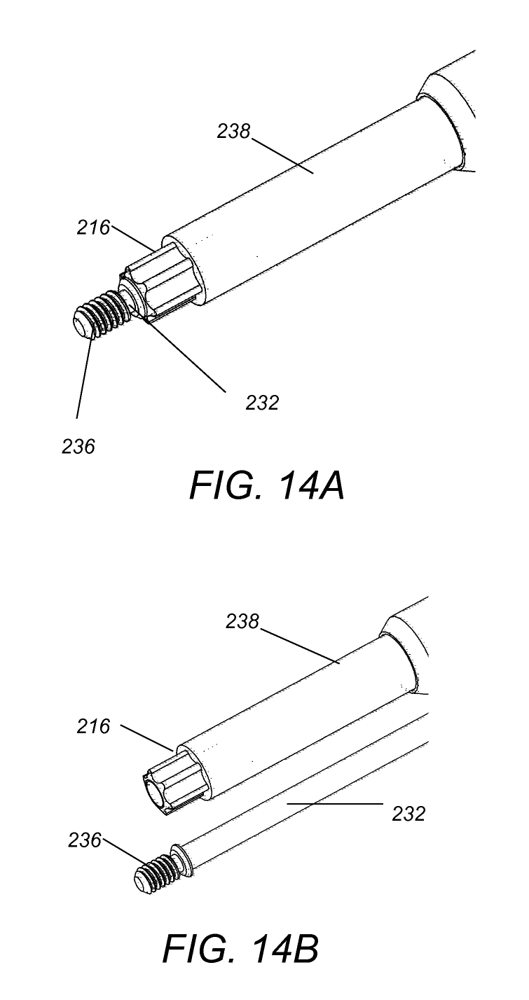

Referring to FIG. 14a, and FIG. 14B, the driver tool 200 includes an inner cylindrical shaft 232 having a screw 236 at its distal end, instead of an inner central blade 226. Screw 236 is used for removing a bone screw from a vertebral location. In this case, opening 128 in the bone screw head top 123 includes inner threads. Inner cylindrical shaft 232 rotates clockwise independently of the outer sleeve 238 and attaches screw 236 to the threaded hole 128, thereby locking the driver tool 200 to the screw 120. Rotating the driver tool 200 counter-clockwise removes the screw 120 from its place.

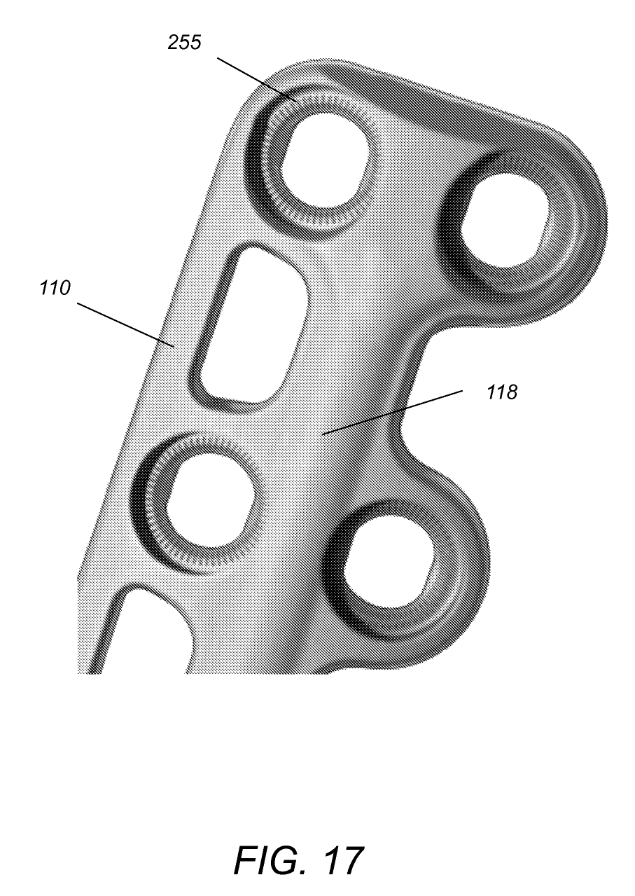

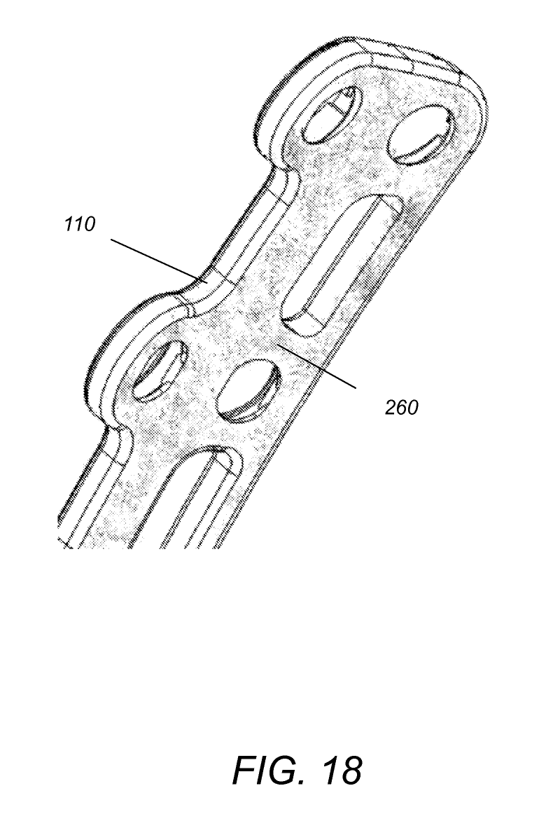

Other embodiments may include the following. The cervical plate 110 may be one-level bone plate configured to stabilize two adjacent vertebras and may have four through-openings 114, shown in FIG. 16. In yet other embodiments, plate 110 may be a three or four level plate stabilizing four or five adjacent vertebras, respectively. The plate 110 may have various lengths in order to provide better interface with the vertebral anatomy. The plate length and/or width may be adjustable. As shown in FIG. 16, cervical plate 110 includes pinholes 151a, 151b for temporary support pins 152 used to hold the plate in place, while it is being fastened down. The bone plate 110 may be made of metal, plastic, ceramic, bone, polymers, composites, absorbable material, biodegradable material, or combinations thereof. In other embodiments the back surface of cervical plate 110 is roughened, as shown in FIG. 18. The roughened surface structure 260 is used for providing a secure grip into the vertebral surfaces. The screw head 122 may be integral or non-integral with the screw main body 124. Opening 128 may have other geometrical shapes including, pentagonal, hexagonal, and octagonal, among others. The flexible arms 121a-121c may be integral or non-integral with the screw head. In yet other embodiments, the flexible arms may extend from the main body 124 of the screw and may be integral or non-integral with the main body 124. The number of flexible arms 121a-121c may be more or less than three. Each arm 121a-121c may be composed of multiple parts. Flexible arms 121a-121c may comprise curved, angled or beveled outer surfaces 129a-129d which cooperate with the corresponding outer surfaces of lip 132 during the driving of the screw 120 into the vertebral location. The flexing of the flexible arms during insertion and the following unflexing of the flexible arms once they are in place, serve as a visual indicator to the user that the screw is fully inserted and engaged into the plate and vertebra. In other embodiments through-openings 114a-114b include laser-etched ridges 255 arranged perpendicular to the groove 133 around the inner wall perimeter of the openings, as shown in FIG. 17. Ridges 255 interface and engage with complementing structures in the outer surface of the cylindrical center 126 of the bone screw head 122 and further prevent the bone screw 120 from rotational and axial movement.

Several embodiments of the present invention have been described. Nevertheless, it will be understood that various modifications may be made without departing from the spirit and scope of the invention. Accordingly, other embodiments are within the scope of the following claims.

What is claimed is:

1. An implantable cervical plate assembly for stabilization of two adjacent spinal vertebras, comprising:
a cervical plate comprising an elongated asymmetric body having a first straight side surface, a second contoured side surface opposite to the first side surface, front and back surfaces and top and bottom surfaces and wherein said elongated asymmetric body comprises two or more through-openings extending from the front surface to the back surface of said elongated asymmetric body;
two or more bone fasteners configured to be inserted through said two or more through-openings, respectively, and attached to two or more locations in said two adjacent spinal vertebras, respectively, thereby attaching said cervical plate to said spinal vertebras;
wherein said through-openings comprise a first diameter at the front surface of said elongated body, a second diameter at the back surface of said elongated body and a third diameter in the area between the front and back surfaces of the elongated body and wherein said first diameter is smaller than said third diameter, thereby forming a lip at the top of said through-openings and wherein said third diameter is larger than the second diameter and said first diameter is larger than the second diameter, thereby forming a groove within the perimeter of the inner wall of said through-openings; and
wherein said bone fasteners comprise a threaded main body and a head and wherein said threaded main body comprises threads for engaging said spinal vertebras and wherein said head comprises one or more flexible structures configured to be flexed and inserted into said groove and then unflex and remain captured within said groove.

2. The assembly of claim 1, wherein said through-openings comprise an oval-shaped perimeter at the back surface and wherein said oval-shaped perimeter comprises two parallel straight sides and two opposite curved sides and wherein the distance between the two parallel straight sides is smaller than the major diameter of the threads of the bone fasteners and wherein the distance between the curved sides is equal to or larger than the major diameter of the threads of the bone fasteners.

3. The assembly of claim 1 wherein said bone fastener head comprises a cylindrical main body and wherein said one or more flexible structures comprise one or more flexible arms extending tangentially from the outer side surface of said cylindrical main body and curving counter-clockwise around the cylindrical main body and wherein the diameter of the bone fastener head including the flexible arms in the unflexed position is larger than the first diameter of said through openings and wherein said flexible arms are configured to flex inward toward the outer side surface of the cylindrical main body when they come in contact with said lip while the bone fastener is rotated clock-wise to be driven into the vertebras and then said flexible arms unflex once they are below the lip.

4. The assembly of claim 1, wherein said bone fastener head comprises an opening extending into said threaded main body and wherein said opening comprises an inner surface having six inward protruding lobes and a bottom having six grooves.

5. The assembly of claim 4 further comprising a driver tool, wherein said driver tool comprises an elongated shaft, a handle attached to the proximal end of the elongated shaft and a bone fastener-engaging component attached to the distal end of the elongated shaft and wherein said bone fastener-engaging component comprises one or more structures that complement and engage at least one of said grooves and lobes of the bone fastener opening, respectively.

6. The assembly of claim 5 wherein said structures of the fastener-engaging component comprise four lobes that complement and engage four of the six lobes of the bone fastener opening and two opposite tubular protrusions configured to be positioned and engage two opposite located grooves of the bone fastener opening.

7. The assembly of claim 6 wherein the fastener-engaging component comprises a driver and a locking sleeve and wherein the driver comprises an elongated cylindrical body having said structures at its distal end and a slot extending along the driver tool axis and wherein said cylindrical body flexes and snaps into said bone fastener opening and wherein said locking sleeve is configured to move down and lock the driver into the bone fastener opening.

8. The assembly of claim 7 wherein said locking sleeve comprises a tubular cylindrical body and a central blade and wherein the tubular cylindrical body is dimensioned to fit and slide over said driver cylindrical elongated body and wherein said central blade is configured to be placed within said driver slot.

9. The assembly of claim 3 wherein said flexible arms comprise curved, angled or beveled outer surfaces and wherein said flexible arms outer surfaces cooperate with matching outer surfaces of said lip.

10. The assembly of claim 1, wherein said bone fastener head comprises an opening extending into said threaded main body and wherein said opening comprises pentagonal, hexagonal or octagonal geometric shape.

11. The assembly of claim 1, wherein said bone fastener head comprises an opening extending into said threaded main body and wherein said opening comprises inner threads.

12. The assembly of claim 1 wherein said cervical plate further comprises one or more elongated openings configured to support bone graft material.

13. The assembly of claim 2 wherein said bone fasteners further comprise a tapered portion extending between the threaded main body and the head and wherein said parallel straight sides of said through-openings cut into the diameter of the tapered portion for a tighter secure lock and fit.

14. The assembly of claim 1, wherein said through-openings further comprise laser-etched ridges extending perpendicular to said groove.

15. The assembly of claim 1 wherein said back surface of the cervical plate comprises a roughened texture.

16. An implantable cervical plate assembly for stabilization of two adjacent spinal vertebras, comprising:
a cervical plate comprising an elongated body having first and second side surfaces, front and back surfaces and top and bottom surfaces and wherein said elongated body comprises two or more through-openings extending from the front surface to the back surface of said elongated body;
two or more bone fasteners configured to be inserted through said two or more through-openings, respectively, and attached to two or more locations in said two adjacent spinal vertebras, respectively, thereby attaching said cervical plate to said spinal vertebras, wherein said bone fasteners comprise a threaded main body and a head and wherein said threaded main body comprises threads for engaging said spinal vertebras;
wherein said through-openings comprise a perimeter dimensioned and shaped to match and complement the shape of said bone fastener head and wherein said through-openings further comprise two opposite radially extending slots and two grooves positioned adjacent to said slots within the inner wall of said through openings, respectively; and
wherein said head comprises two opposite radially protruding tubular extensions dimensioned and configured to be inserted into said two opposite radially extending slots and then rotated and captured within the two adjacent grooves, respectively.

17. A bone fastener driver tool comprising:
an elongated shaft,
a handle attached to the proximal end of the elongated shaft; and
a bone fastener-engaging component attached to the distal end of the elongated shaft and wherein said bone fastener-engaging component comprises one or more structures that complement and engage at least one of grooves and protruding lobes within an opening of a bone fastener, respectively;
wherein the bone fastener-engaging component further comprises a driver and a locking sleeve and wherein the driver comprises an elongated cylindrical body having said structures at its distal end and a slot extending along the driver tool axis and wherein said cylindrical body flexes and snaps into said bone fastener opening and wherein said locking sleeve is configured to move down and lock the driver into the bone fastener opening.

18. The bone fastener driver tool of claim 17 wherein said one or more structures of the bone fastener-engaging component comprise four lobes that complement and engage four lobes in the bone fastener opening and two opposite tubular protrusions configured to be positioned and engage two opposite located grooves in the bone fastener opening.

19. The bone fastener driver tool of claim 18 wherein said locking sleeve comprises a tubular cylindrical body and a central blade and wherein the tubular cylindrical body is dimensioned to fit and slide over said driver cylindrical elongated body and wherein said central blade is configured to be placed within said driver slot.

20. The bone fastener driver tool of claim 17 wherein said one or more structures comprise outer threads that engage inner threads formed within the bone fastener opening.

21. A method for stabilizing two adjacent spinal vertebras, comprising:
providing a cervical plate comprising an elongated asymmetric body having a first straight side surface, a second contoured side surface opposite to the first side surface, front and back surfaces and top and bottom surfaces and wherein said elongated asymmetric body comprises two or more through-openings extending from the front surface to the back surface of said elongated asymmetric body;
inserting two or more bone fasteners through said two or more through-openings, respectively, and attaching them to two or more locations in said two adjacent spinal vertebras, respectively, thereby attaching said cervical plate to said spinal vertebras;
wherein said through-openings comprise a first diameter at the front surface of said elongated body, a second diameter at the back surface of said elongated body and a third diameter in the area between the front and back surfaces of the elongated body and wherein said first diameter is smaller than said third diameter, thereby forming a lip at the top of said through-openings and wherein said third diameter is larger than the second diameter and said first diameter is larger than the second diameter, thereby forming a groove within the inner wall of said through-openings; and
wherein said bone fasteners comprise a threaded main body and a head and wherein said threaded main body comprises threads for engaging said spinal vertebras and wherein said head comprises one or more flexible structures configured to be flexed and inserted into said groove and then unflex and remain captured within said groove.

22. The method of claim 21, wherein said through-openings comprise an oval-shaped perimeter at the back surface and wherein said oval-shaped perimeter comprises two parallel straight sides and two opposite curved sides and wherein the distance between the two parallel straight sides is smaller than the major diameter of the threads of the bone fasteners and wherein the distance between the curved sides is equal to or larger than the major diameter of the threads of the bone fasteners.

23. The method of claim 21 wherein said bone fastener head comprises a cylindrical main body and wherein said one or more flexible structures comprise one or more flexible arms extending tangentially from the outer side surface of said cylindrical main body and curving counter-clockwise around the cylindrical main body and wherein the diameter of the bone fastener head including the flexible arms in the unflexed position is larger than the first diameter of said through openings and wherein said flexible arms are configured to flex inward toward the outer side surface of the cylindrical main body when they come in contact with said lip while the bone fastener is rotated clock-wise to be driven into the vertebras and then said flexible arms unflex once they are below the lip.

24. The method of claim 21, wherein said bone fastener head comprises an opening extending into said threaded main body and wherein said opening comprises an inner surface having six inward protruding lobes and a bottom having six grooves.

25. The method of claim 24 further comprising providing a driver tool, wherein said driver tool comprises an elongated shaft, a handle attached to the proximal end of the elongated shaft and a bone fastener-engaging component attached to the distal end of the elongated shaft and wherein said bone fastener-engaging component comprises one or more structures that complement and engage at least one of said grooves and lobes of the bone fastener opening.

26. The method of claim 25 wherein said structures of the fastener-engaging component comprise four lobes that complement and engage four of the six lobes of the bone fastener opening and two opposite tubular protrusions configured to be positioned and engage two opposite located grooves of the bone fastener opening.

27. The method of claim 26 wherein the fastener-engaging component comprises a driver and a locking sleeve and wherein the driver comprises an elongated cylindrical body having said structures at its distal end and a slot extending along the driver tool axis and wherein said cylindrical body flexes and snaps into said bone fastener opening and wherein said locking sleeve is configured to move down and lock the driver into the bone fastener opening.

28. The method of claim 27 wherein said locking sleeve comprises a tubular cylindrical body and a central blade and wherein the tubular cylindrical body is dimensioned to fit and slide over said driver cylindrical elongated body and wherein said central blade is configured to be placed within said driver slot.
